(19) United States
(12) Patent Application Publication
(10) Pub. No.: US 2021/0162380 A1
(43) Pub. Date: Jun. 3, 2021

(54) AROMATIZATION CATALYST WITH IMPROVED ISOMERIZATION, PROCESS OF MAKING AND USING THEREOF

(71) Applicant: SABIC GLOBAL TECHNOLOGIES B.V., Bergen op Zoom (NL)

(72) Inventors: Travis CONANT, Richmond, TX (US); Dustin FARMER, Houston, TX (US); Alla KHANMAMEDOVA, Sugar Land, TX (US); Neeta KULKARNI, Houston, TX (US); Scott STEVENSON, Houston, TX (US); Jun WANG, Sugar Land, TX (US)

(21) Appl. No.: 17/267,109

(22) PCT Filed: Aug. 30, 2019
(86) PCT No.: PCT/IB2019/057347

§ 371 Date: Feb. 9, 2021

Related U.S. Application Data
(60) Provisional application No. 62/725,001, filed on Aug. 30, 2018.

(51) Int. Cl. B01J 29/44; B01J 37/02; B01J 37/30

(52) U.S. Cl. CPC B01J 29/44; B01J 37/30; B01J 37/0201

(57) ABSTRACT

Disclosed is a catalyst comprising a zeolite comprising a framework, the framework comprising silicon and aluminum, and a noble metal. The zeolite has undergone at least a first exchange with a Group I or II cation or ammonium and thereafter is contacted with a second Group I or II cation. The step of contacting comprises an exchange, incipient wetness, or dry impregnation. The noble metal is deposited at the zeolite.

### TECHNICAL FIELD

The disclosure generally relates to the process for the aromatization of alkanes to aromatics, and more specifically to the use of a zeolite catalyst for the process for the aromatization of alkanes to aromatics.

### BACKGROUND

The aromatization of alkanes is a multi-step process of dehydro-cyclization. Appropriate catalysts for this process are typically multi-functional to provide acceptable conversion and selectivity for the desired products. Zeolites are known catalysts for a number of reactions including isomerization, toluene disproportionation, transalkylation, hydrogenation and alkane oligomerization, and aromatization.

U.S. Pat. No. 6,784,333 hereby incorporated by reference, discloses a catalyst of an aluminum-silicon germanium zeolite on which platinum has been deposited. The catalyst can be used in aromatization of alkanes, specifically, aromatization of lower alkanes, such as propane. The catalyst may be a MFI zeolite in which germanium is incorporated into the crystalline framework, that is, platinum/germanium Zeolite Socony Mobil-5 Pt/Ge-ZSM-5. The catalyst may be sulfided before or during the aromatization process.

U.S. Pat. No. 8,722,950 discloses processes for producing alkenes (such as propylene and hexene) by metathesis from butenes and pentenes and subsequent aromatization of generated hexanes. The metathesis catalyst may comprise transition metal compounds of tungsten, molybdenum, or rhenium. The aromatization catalysts may comprise zeolites having a Group VIII deposited metal, such as platinum, and elements other than silicon Si and aluminum Al, such as germanium Ge, in the zeolite crystalline framework.

U.S. Pat. No. 7,902,413 discloses non-acidic ZSM-5 zeolite with germanium incorporated into the framework of the zeolite. The zeolite is made non-acidic by being base-exchanged with an alkali metal or alkaline earth metal.

U.S. Pat. Pub. No. 2016/0288108 discloses a catalyst comprises: a zeolite comprising Si, Al, and Ge in the framework with Pt deposited thereon; wherein the catalyst has an silicon:aluminum Si:Al₂mole ratio of greater than or equal to 125, an Si:Ge mole ratio of 40 to 400, and an sodium: aluminum Na:Al mole ratio of 0.9 to 2.5, wherein the catalyst has an aluminum content of less than or equal to 0.75 wt. %, wherein the catalyst is non-acidic.

Conventional aromatization catalysts and technologies however generally require six linear carbons to form benzene and higher aromatics. Accordingly, the conversion of alkane feeds comprising branched/non-linear alkanes may be very low or negligible at best. For example, the industrial conversion of naphtha or shale gas condensate feeds to aromatic compounds is limited because of the considerable amount of iso-hexanes within the feeds. In other words, conventional catalyst performance may be limited by the amount of iso-hexanes in the feed because the iso-hexanes are essentially inert and do not convert to aromatics. As the feed becomes more branched the overall aromatic yield may decrease as a large amount of the feed goes unreacted. While isomerization catalysts are understood to require acidity to facilitate a skeletal rearrangement, acidity in aromatization catalysts may result in undesirable cracking.

Therefore, there is a need for aromatization catalysts with activity for skeletal isomerization with minimum cracking activity.

### SUMMARY

Aspects of the present disclosure relate to a catalyst. The catalyst may comprise a zeolite comprising a framework and a noble metal deposited at the framework. The framework may comprise silicon and aluminum. The zeolite has undergone at least a first exchange with a Group I or II cation or ammonium and is thereafter is contacted with a second Group I or II cation. The step of contacting comprises an exchange, incipient wetness, or dry impregnation. The noble metal may be deposited at the zeolite after the step of contacting.

In further aspects, the present disclosure relates to a catalyst comprising a zeolite comprising a framework and a noble metal deposited at the framework. The framework of the zeolite may comprise silicon and aluminum. The zeolite may further comprise cesium and sodium ions.

Further aspects relate to methods of forming the disclosed catalyst.

### BRIEF DESCRIPTION OF THE DRAWINGS

The above-mentioned and other features and advantages of this disclosure, and the manner of attaining them, will become apparent and be better understood by reference to the following description of one aspect of the disclosure in conjunction with the accompanying drawings, wherein:

FIG. 1 shows the benzene yield for Catalysts A, C, and D at varying isomer to normal hexane (nc6) ratios.

FIG. 2 shows the space time yield for tonnes of benzene per tonne of catalyst for Catalysts A, C, and D.

### DETAILED DESCRIPTION

The present disclosure can be understood more readily by reference to the following detailed description of the disclosure and the Examples included therein. It is also to be understood that the terminology used herein is for the purpose of describing particular aspects only and is not intended to be limiting.

Various combinations of elements of this disclosure are encompassed by this disclosure, that is, combinations of elements from dependent claims that depend upon the same independent claim.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

Alkane aromatization is a multistep process, which usually features multifunctional catalysts such as zeolite based catalytic systems or catalysts comprising a hydrogenation metal loaded onto a zeolite. Conventional aromatization catalysts and technologies however generally require six linear carbons to form benzene and higher aromatics. As a result, the yield for the conversion of alkane feeds having branched/non-linear alkanes may be negligible. For example, a naphtha or shale gas condensate feed having considerable amounts of iso-hexanes is limited in its benzene yield by the amount of iso-hexanes within the feed. As the feed becomes more branched the overall aromatic yield may decrease as a large amount of the feed goes unreacted. To alleviate this, conventional catalysts having acidity may be to facilitate skeletal isomerization. However, the acidic catalyst for aromatization are also known to cause the feed to break down into smaller, hydrocarbons (“cracking”). The present disclosure provides aromatization catalysts that have activity for skeletal isomerization as well as the process of converting a feed that contains both branched and linear C6⁺ hydrocarbons to benzene and higher aromatics. As provided herein, the disclosed catalysts may perform by converting iso-hexane to n-hexane in-situ and subsequently converting n-hexane to benzene, thereby increasing the overall aromatization yield.

In various aspects, the present disclosure provides a catalyst derived from a zeolite comprising a framework of silicon, aluminum, and a noble metal, where the zeolite underdoes a first exchange with a first Group I or II cation or an ammonium ion and is thereafter contacted with a second Group I or II cation. The first and second Group I or II cation may be the same or different elements. The step of contacting may comprise an exchange, incipient wetness, or dry impregnation.

The disclosed catalyst comprises platinum on an aluminum-silicon-germanium zeolite (GeZSM-5) that has been treated with alkali metals. In various aspects of the present disclosure, a catalyst of GeZSM-5 undergoes at least a first exchange with an alkali metal (or an alkaline earth metal) or an ammonium ion and one or more calcining processes. A noble metal may be deposited at the zeolite. In further aspects, the GeZSM-5 zeolite undergoes a first ion exchange and a second ion exchange. The disclosed catalysts are appropriate for the conversion of C₆₊, for example, C₆-C₁₂ alkanes to aromatics. The amounts and methods of modifying the GeZSM-5 with the alkali metals permit tuning isomerization activity.

The disclosed catalyst may begin with a GeZSM-5 and may be modified with cations to tune the isomerization activity while concurrently minimizing unwanted cracking of the hydrocarbon feed. Conventional catalysts, such as the catalyst described in U.S. Pat. No. 7,901,413, may achieve increased selectivity to benzene and higher aromatics from a C₆₊ feed, the catalyst when completely base-exchanged with alkali metals may have a negligible isomerization activity (such as, for example, 4% conversion). Via a deliberate process of exchange of an alkali metal or mixtures of alkali metals, the disclosed catalysts increase the isomerization conversion dramatically while maintaining the high selectivity (that is, low cracking activity) of the conventional aromatization catalysts at values greater than 90%.

The disclosed catalysts may be useful for a broader range of feeds than conventional options because they can convert a substantial fraction of the iso-hexane within the feed to n-hexane and subsequently to benzene where the iso-hexane goes unconverted when using a conventional catalyst. Moreover, because the skeletal isomerization of hexanes may be limited by equilibrium, the amount of n-hexane that may be made is also typically limited if one were to perform isomerization in a separate unit. The disclosed catalysts eliminate the need for a separate unit to for isomerization by facilitating the isomerization and aromatization in a common reactor thereby giving a sink for n-hexane. In other words, as n-hexane is formed via isomerization it is aromatized, thereby overcoming equilibrium limits by consuming n-hexane.

Catalysts of the present disclosure may be derived from a zeolite. Crystalline silicates, aluminosilicates, aluminophosphates and silicoaluminophosphates have uniform pores through which molecules can diffuse. Aluminosilicates include zeolites. Examples of zeolites are MFI (ZSM-5), BEA (Beta), MWW (MCM-22), MOR (Mordenite), LTL (Zeolite L), MTT (ZSM-23), MTW (ZSM-12), TON (ZSM-22) and MEL (ZSM-11). Crystalline silicates, aluminosilicates, aluminophosphates and silicoaluminophosphates have structures consisted of TO₄ tetrahedra, which form a three dimensional network by sharing oxygen atoms where T represents tetravalent elements, such as silicon; trivalent elements, such as aluminum; and pentavalent elements, such as phosphorus. “Zeolites” may comprise aluminosilicates with open three-dimensional framework structures composed of corner-sharing TO₄ tetrahedra, where T is Al or Si, but also includes tetravalent, trivalent and divalent T atoms which able to isoelectronically replace Si and Al in the framework, e.g., germanium(4⁺), titanium (4⁺), boron (3⁺), gallium (3⁺), iron (3⁺), zinc (2⁺) and beryllium (2⁺). “Zeolite” is primarily a description of structure, not composition. The crystalline framework of which these elements from Group IV, Group V, Group XIII, Group XIV, Group XV and the first series transition metals are part and on which metals selected from Group VI, Group VII, Group VIII, Group XIV and Group X are deposited need not be limited to a zeolite but may be any microporous silicate, aluminosilicate, aluminophosphate or silicoaluminophosphate. In one example, the catalyst comprises germanium in an aluminosilicate framework (a Ge-zeolite).

In various aspects, the zeolite framework comprises a metalloid. In one example, the metalloid comprises germanium. Besides germanium, there may be other elements in the crystalline framework which associate with platinum or other deposited metals. Elements in the crystalline framework may be selected from Group IV, Group V, Group XIII, Group XIV, Group XV and the first series transition metals of the Periodic Table of Elements. Examples of these elements are germanium, boron, gallium, indium, tin, titanium, zirconium, vanadium, chromium, iron, niobium and phosphorus. One or more elements may be in the crystalline framework. In further aspects, the metalloid is optional. Suitable Group III elements include boron B, aluminum Al, and gallium Ga etc are in Group III; while suitable group IV elements include carbon C, silicon Si, germanium Ge, and tin Sn.

The metalloid, for example germanium, content of the zeolite framework may be from about 0.2% to about 10% by weight. For example, the germanium content of the zeolite may be from 2% to 8% by weight or from 2% to 6% by weight of the zeolite.

A Ge-zeolite of the present disclosure may be synthesized from an aqueous gel containing a silica source, a germanium source, an aluminum source and a structure directing agent. A technique for synthesizing Ge-zeolites may comprise converting an aqueous gel of a silica source, a germanium source and an aluminum source to zeolite crystals by a hydrothermal process, employing a dissolution/recrystallization mechanism. The reaction medium may also contain structuring agents which are incorporated in microporous space of the zeolite network during crystallization, thus controlling the construction of the network and assisting to stabilize the structure through the interactions with the zeolite components. The reaction mixture gel may be heated and stirred to form zeolite crystals and then cooled. The zeolite crystals may be separated from the gel and washed, dried and calcined.

Silicates, aluminosilicates, aluminophosphates and silicoaluminophosphates generally crystallize from an aqueous solution. A typical technique for synthesizing silicates, aluminosilicates, aluminophosphates or silicoaluminophosphates comprises converting an aqueous gel of a silica source, an aluminum source and a phosphorus source, as needed, to crystals by a hydrothermal process, employing a dissolution/recrystallization mechanism. The reaction medium may also contain an organic structure-directing agent which is incorporated in the microporous space of the crystalline network during crystallization, thus controlling the construction of the network and assisting to stabilize the structure through the interactions with the silicon, aluminum or phosphorus components.

The aqueous gel contains in addition to the silica source, the aluminum source, the phosphorus source, as needed, and the optional organic structure-directing agent, and a source of at least one other element from Group IV, Group V, Group XIII, Group XIV, Group XV or the first series transition metals to be incorporated into the framework of the silicate, aluminosilicate, aluminophosphate or silicoaluminophosphate.

Examples of the silica source are silicon oxide or silica (SiO₂) which is available in various forms, such as silica sol, commercially available as Ludox AS40™, precipitated silica, commercially available as Ultrasil VN3SP™ and fumed silica, commercially available as Aerosil 200™. Examples of the aluminum source include sodium aluminate, aluminum nitrate, aluminum sulfate, aluminum hydroxide and pseudobohemite. Examples of the phosphorus source are phosphoric acid (85 wt. %), P₂O₅, orthophosphoric acid, triethylphosphate and sodium metaphosphate. Examples of the source of Group IV, Group V, Group XIII, Group XIV, Group XV and the first series transition metals are oxides, chlorides, sulfates, alkoxides, fluorides, nitrates and oxalates.

Examples of the structure-directing agent are organic amine and quaternary ammonium compounds and salts and cations thereof, including but not limited to tetra n-propyl ammonium hydroxide, tetra n-propyl ammonium bromide and tetra n-propyl ammonium chloride, and tetraethyl ammonium hydroxide.

The zeolite may have particular silicon to aluminum atomic ratio (Si:Al₂). For example, the Si:Al₂ of the zeolite may be greater than 2. One example, intending to be limiting, is a Si:Al₂ atomic ratio in the range from 10 to 200. Another example, without limiting the disclosure, is a Si:Al₂ atomic ratio in the range from 20 to 300 or from 20 to 150 or from 20 to 110.

Further description of the making of catalyst described herein are presented. A zeolite, such as GeZSM-5, may be subjected to an ion-exchange with a cation. The cation may be a hydrogen cation or a Group I or Group II cation (an alkali or alkaline earth metal), such as cesium. In further examples, the cation may be ammonium NH₄⁺. The ion-exchange may be followed by a second exchange with another Group I or Group II element, for example, an alkali metal such as sodium. In certain aspects, a calcining process may follow the initial ion exchange or may follow the initial ion exchange as well as the back ion-exchange with the Group I or Group II element. For example, a zeolite, such as GeZSM-5, may be subjected to an ion-exchange with cesium. The ion-exchanged zeolite may be back exchanged with sodium. The back exchanged zeolite may be impregnated with a noble metal such as platinum. In a further example, a zeolite, such as GeZSM-5, may be subjected to an ion-exchange with ammonium cation. The ion-exchanged zeolite may be contacted with sodium. The ion-exchanged zeolite may be impregnated with a noble metal such as platinum. In certain aspects, a calcining process may follow the initial ion exchange.

The zeolite may be subjected to at least one ion exchange. Ion-exchange may take place during synthesis of the zeolite with an alkali metal or alkaline earth metal being added as a component of the reaction mixture or may take place with a crystalline zeolite before or after deposition of the noble metal. The zeolite may undergo at least one exchange with an alkali Group I or II element or with an ammonium ion. As an example, the alkali Group I or II element may comprise cesium. Where the zeolite is subjected to an exchange with an alkali Group I or II element, the ion-exchanged zeolite may be contacted with a second alkali Group I or II element. The alkali Group I or II element for the ion exchange and contacting may be different elements. As an example, the exchange may be performed with cesium as the alkali metal ion and the ion-exchanged zeolite may be contacted with a sodium ion. The contacting may be performed in a number of ways to effect the addition of an ion that provides an ion-exchanged zeolite or a zeolite containing said ion. For example, the ion exchanged zeolite may be contacted with a second Group I or II element via an ion exchange process, an incipient wetness process, or a dry impregnation process. In one example, the ion-exchanged zeolite may undergo a second ion-exchange. As a more specific example, the second alkali Group I or Group II cation or element may comprise sodium. It is noted that the first alkali Group I or Group II cation may be the same element or a different element from the second alkali Group I or Group II cation.

As provided above, the disclosed catalyst comprises a zeolite that has undergone at least one exchange with a Group I or II cation. For example, the disclosed catalyst comprises a zeolite that has undergone at least a first exchange with an alkali metal or alkaline earth metal. These exchange elements or metals may comprise cesium, potassium, sodium, rubidium, barium, calcium, magnesium and combinations thereof. An ion exchange, or a base exchange may result in the zeolite being non-acidic. The zeolite may be base-exchanged to the extent that most or all of the cations associated with aluminum are alkali metal or alkaline earth metal. An example of a monovalent base: aluminum molar ratio in the zeolite after ion exchange is at least 0.7 or at least about 0.9. In further examples, 50% of the cation of the first exchange is replaced by the Group I or II cation when the zeolite thereafter is contacted with a second Group I or II cation. In yet further examples, at least 50% , or for example, at least 90%, at least 95%, at least 99%, or at least 99.9% by weight of the cation of the first exchange is replaced by the second Group I or II cation when the zeolite thereafter is contacted with a Group I or II cation. The zeolite may exhibit a molar ratio of alkali metal to alumina in the zeolite framework of from 0.7 to 1.5 as a result of contacting the zeolite with a Group I or II element.

To provide the disclosed catalyst, the ion-modified zeolite may be calcined at different stages in the synthesis. Calcination may proceed at a temperature in the range of from about 300° C. to about 1000° C. for a duration of about one hour to about 24 hours. Calcination may also occur after metal deposition to fix the metal. This calcination may be at a temperature of about 200° C. to about 550° C. for a time in the range of from about 0.5 hour to about 24 hours. Calcination may occur in an environment of oxygen, nitrogen, hydrogen, helium and mixtures thereof For example, there may be a calcining step after ion-exchange at the zeolite with an alkali Group I or II element, of about 280° C., or with an ammonium ion, of about 550° C. Where the zeolite is subjected to an exchange with an alkali Group I or II element and the ion-exchanged zeolite is contacted with a second alkali Group I or II element, a second calcining step may occur. The second calcining step may proceed at about 280° C. The disclosed calcining may occur after a noble metal is deposited at the zeolite. The zeolite may contain promoters or modifiers as are known in the art. These promoters or modifiers are present in a catalytically effective amount, for example., about 0.1 weight percent (wt. %) to about 1.0 weight percent.

As provided herein, the catalyst may comprise a noble metal disposed, or impregnated, at the zeolite. Noble metals may comprise metals such as platinum, palladium, iridium, rhodium and ruthenium for example. Deposited metals may be selected from Group VIII, Group IX and Group X of the Periodic Table of Elements. Specific examples of the deposited metal are platinum, molybdenum, rhenium, nickel, ruthenium, rhodium, palladium, osmium and iridium. One or more metals, such as bimetallics, e.g., platinum/tin Pt/Sn, platinum/germanium Pt/Ge, platinum/lead Pt/Pb or metal/metal oxide combinations, for example. platinum germanium oxide Pt/GeO₂, may be deposited. In a specific example, the noble metal comprises platinum. The noble metal may deposited on the zeolite by any known method of depositing a metal on a zeolite. Typical methods of depositing a metal on zeolite are ion exchange and impregnation. Deposition of the noble metal results in the noble metal being present not only on the surface of the zeolite but also in the pores and channels of the zeolite.

In one example of the present disclosure, the noble metal may present in the catalyst in the range from 0.05% to 3% by weight. In another example of the present disclosure, the noble metal is from 0.2% to 2% by weight. In another example, the noble metal is from 0.2 to 1.5% by weight. In a further example, platinum is present in the catalyst in the range of from 0.05% to 3%, percent by weight. The zeolite may be impregnated with 1 wt. % platinum.

In certain aspects, the catalyst may be subjected a pretreatment or regeneration step. The pretreatment or regeneration step may be used to improve selectivity of the catalyst. As an example, the catalyst may be chlorinated or oxychlorinated as a pretreatment or regeneration step.

Also presented herein are methods of forming aromatics from alkanes using the disclosed catalysts. The method may comprise contacting an alkane-containing feedstream with the catalyst at temperatures of from about 400° C. to about 730° C. The catalyst may comprise the disclosed ion exchanged zeolite.

The disclosed catalyst may be useful for the conversion of C₆-C₁₂ alkanes to aromatics. These alkanes may comprise alkanes such those that might be obtained from natural gas condensate, light naphtha, raffinate from aromatics extraction and other refinery or chemical processes. In one example, the disclosed catalysts may facilitate the conversion of naphtha. The disclosed catalysts are useful for the conversion to aromatics of iso- and regular C₆₊ alkanes. In a specific example, the disclosed catalysts are useful for conversion to aromatics of C₆-C₁₂ alkanes having fewer than six linear carbons. These aromatics may include, for example, benzene, ethyl benzene, toluene and xylenes.

The present alkane aromatization process may comprise directing a feedstream comprising an alkane containing six to twelve carbon atoms (C₆-C₁₂) to a reactor. In some examples, the feedstream may comprise an alkane and a diluent such that alkanes having five carbon atoms may be present. The alkane-containing feedstream may be contacted with the disclosed catalyst to promote the reaction of C₆-C₁₂ alkanes to aromatic hydrocarbons such as benzene at a temperature of about 400° C. to about 730° C. and a pressure of about 0.01 to about 1.0 megaPascals MPa. The primary desired products of the process of this disclosure are benzene, toluene, ethyl benzene and xylene.

The alkane-containing feed as described herein may be under such conditions as: at a weight hourly space velocity of 1-500 per hour (h⁻¹), or more specifically at 1 h⁻¹ to 100 h⁻¹; at a temperature between 250° C. and 800° C., or more specifically from about 400° C. to about 700° C.

Aspects of the present disclosure provide aromatization of alkanes using the disclosed catalysts, where the alkanes have between six and twelve carbon atoms per molecule, to produce aromatics, such as benzene, ethyl benzene, toluene and xylene. The contact between the alkanes and the catalyst may proceed at a liquid hourly space velocity in a range between 0.1 h⁻¹ and 100 h⁻¹, at a temperature greater than or equal to 450° C., and at a pressure in a range between 0.03 MPa and 2.17 MPa. In other aspects, the contact between the alkanes and the catalyst is at a liquid hourly space velocity in a range between 0.1 h⁻¹, and 100 h⁻¹, at a temperature in a range between 450° C. and 650° C. and at a pressure in a range between 0.03 MPa and 2.17 MPa. In further aspects, the contact between the alkanes and the catalyst may be at a liquid hourly space velocity in a range between 0.1 h⁻¹ and 100 h⁻¹, at a temperature in a range between 450° C. and 625° C. and at a pressure in a range between 0.03 MPa and 2.17 MPa. The contact between the alkanes and the catalyst is at a liquid hourly space velocity in a range between 0.1 h⁻¹ and 100 h⁻¹, at a temperature in a range between 450° C. and 600° C. and at a pressure in a range between 0.03 MPa and 2.17 MPa. The contact between the alkanes and the catalyst may be at a liquid hourly space velocity in a range between 0.1 h⁻¹ and 100 h⁻¹, at a temperature in a range between 450° C. and 575° C. and at a pressure in a range between 0.03 MPa and 2.17 MPa.

The reactor system for effecting the aromatization process may not be critical. Suitable reactor systems may comprise a fixed bed system, moving bed system or fluidized bed system. The reactor system may be a one-pass system or recycling system. In one aspect, the reactor comprises a zone, vessel, or chamber containing catalyst particles through which the alkane-containing feedstream flows and the reaction takes place. The reactor system may involve a fixed, moving, or fluidized catalyst bed for example. The reaction products then flow out of the bed and be collected. The reaction products may be separated and C6⁺ aromatic hydrocarbons are recovered. Optionally, methane and hydrogen are recovered and optionally the C₅-C₆ hydrocarbons are recycled to the reactor feedstream.

### Properties

The catalyst may be used in a process of aromatization of alkanes, such as alkanes having six to twelve carbon atoms per molecule, to produce aromatics, such as benzene, ethyl benzene, toluene and xylene. The contact between the alkane feed and the catalyst is at a liquid hourly space velocity in the range between 1 h⁻¹ to 2 h⁻¹ at a screening condition of 8.6 at a temperature in the range between 400° C. and 730° C. and at a pressure in the range between 0.1 MPa to 1.0 MPa.

The disclosed developments may produce higher productivity, longer catalyst life, adaptability to certain feedstreams and/or cost reduction in raw material, equipment and process operation. One example of a feedstream to which this catalyst of the present disclosure would be adaptable would be a feedstream which is predominantly, that is, greater than about 50% by volume, iso-hexanes. In one aspect of the present disclosure, the feedstream contains C₅-C₁₂ alkanes or C₅-C₈ alkanes, either alone or as components in a mixture, i.e., in a range from 0% to 100% for each C₅, C₆, C₇ and C₈ alkane.

The disclosed catalysts may be useful for a broader range of feeds than conventional options because the disclosed catalysts can convert a substantial fraction of the iso-hexane within the feed to n-hexane and subsequently to benzene where the iso-hexane goes unconverted when using a conventional catalyst. Moreover, because the skeletal isomerization of hexanes may be limited by equilibrium, the amount of n-hexane that may be made is also typically limited if one were to perform isomerization in a separate unit. The disclosed catalysts eliminate the need for a separate unit to for isomerization by facilitating the isomerization and aromatization in a common reactor thereby giving a sink for n-hexane. In other words, as n-hexane is formed via isomerization it is aromatized, and equilibrium limits may be overcome within the reactor as n-hexane is consumed.

The present disclosure relates at least to the following aspects.

Aspect 1. A catalyst comprising: A zeolite comprising a framework, the framework comprising silicon and aluminum, and a noble metal, wherein (a) the zeolite has undergone at least a first exchange with a Group I or II cation or ammonium cation and (b) the zeolite thereafter is contacted with a second Group I or II cation, wherein the step of contacting comprises an exchange, incipient wetness, or dry impregnation, and wherein the noble metal is deposited at the zeolite after the step of contacting.

Aspect 1B. A catalyst consisting essentially of: A zeolite comprising a framework, the framework consisting essentially of silicon and aluminum, and a noble metal, wherein (a) the zeolite has undergone at least a first exchange with a Group I or II cation or ammonium cation and (b) the zeolite thereafter is contacted with a second Group I or II cation, wherein the step of contacting comprises an exchange, incipient wetness, or dry impregnation, and wherein the noble metal is deposited at the zeolite after the step of contacting.

Aspect 1C. A catalyst consisting of: A zeolite comprising a framework, the framework consisting of silicon and aluminum, and a noble metal, wherein (a) the zeolite has undergone at least a first exchange with a Group I or II cation or ammonium cation and (b) the zeolite thereafter is contacted with a second Group I or II cation, wherein the step of contacting comprises an exchange, incipient wetness, or dry impregnation, and wherein the noble metal is deposited at the zeolite after the step of contacting.

Aspect 2. A catalyst comprising: a zeolite comprising a framework, the framework comprising silicon and aluminum, and a noble metal deposited at the zeolite, wherein the zeolite further comprises cesium and sodium ions.

Aspect 3. The catalyst of any of aspects 1A-2 wherein the framework further comprises a metalloid.

Aspect 4. The catalyst of any of aspects 1A-3, wherein the framework further comprises germanium.

Aspect 5. The catalyst of aspect 4, wherein the zeolite comprises from about 0.2 wt. % to about 10 wt. % germanium.

Aspect 6. The catalyst of any of aspects 1A-5, wherein the zeolite is MFI, BEA, MOR, or LTL.

Aspect 7. The catalyst of any of aspects 1A-5, wherein the zeolite is ZSM-5.

Aspect 8. The catalyst of any of aspects 1A-7, wherein the cation is an ammonium ion or a Group I or Group II alkali cation.

Aspect 9. The catalyst of any of aspects 1A-8, wherein the Group I or II cation is cesium.

Aspect 10. The catalyst of any of aspects 1A-8, wherein the Group I or II cation is sodium.

Aspect 11. The catalyst of any of aspects 1A-10, wherein the zeolite exhibits a molar ratio of alkali metal to alumina in the zeolite framework of from 0.7 to 1.5 as a result of contacting the zeolite with a Group I or II cation in step (b).

Aspect 12. The catalyst of any of aspects IA-C and 3-11, wherein more than 50% of the cation is replaced by the Group I or II cation when the zeolite thereafter is contacted with a Group I or II cation in step (b).

Aspect 13. The catalyst of any of aspects 1 A-C and 3-12, wherein the noble metal comprises platinum.

Aspect 14. The catalyst of any of aspects 3-17, wherein the noble metal is present in an amount of from 0.02 wt. % to 2 wt. %.

Aspect 15. The catalyst of any of aspects 1A-12, wherein the noble metal is present in an amount of from 0.2 wt. % to 2 wt. % based on the total weight of the catalyst.

Aspect 16. The catalyst of any of aspects 3-14, wherein the zeolite undergoes calcination.

Aspect 17. The catalyst of any of aspects 3-15, wherein the zeolite undergoes calcination at a temperature of from about 280° C. to 550° C.

Aspect 18. The catalyst of any of aspects 3-16, wherein the noble metal comprises platinum.

Aspect 19. The catalyst of any of aspects 3-18, wherein the catalyst is suitable for facilitating aromatization of alkanes to aromatics, wherein the alkanes have six to twelve carbons.

Aspect 20. The catalyst of any of aspects 1-19, wherein the catalyst is suitable for facilitating aromatization of alkanes to aromatics and wherein the alkanes comprise isohexanes.

Aspect 21. The catalyst of any of aspects 1A-20, wherein the catalyst provides an aromatic selectivity of at least 90% for conversion of a naphtha feed at 50 hours time on stream for a 0.75 hydrogen/hexane stream at a temperature of about 515° C., atmospheric pressure, and a liquid hourly space velocity of about 8.6 per hour.

Aspect 22. The catalyst of any of aspects 1A-21, wherein the catalyst provides an aromatic selectivity of from about 85% to 99% for conversion of a naphtha feed at 50 hours time on stream for a 0.75 hydrogen/hexane stream at a temperature of about 515 ° C., atmospheric pressure, and a liquid hourly space velocity of about 8.6 per hour.

Aspect 23. The catalyst of any of aspects 1A-21, wherein the catalyst provides a selectivity of about 90% to 99% for conversion of a naphtha feed at 50 hours time on stream for a 0.75 hydrogen/hexane stream at a temperature of about 515° C., atmospheric pressure, and a liquid hourly space velocity of about 8.6 per hour.

Aspect 24. The catalyst of any of aspects 1A-21, wherein the catalyst provides a conversion rate of at least 20% for conversion of a naphtha feed at 50 hours time on stream for a 0.75 hydrogen/hexane stream at a temperature of about 515° C. and a liquid hourly space velocity of about 8.6 per hour when a product is analyzed via gas chromatography.

Aspect 25. The catalyst of any of aspects 1A-21, wherein the catalyst provides a conversion rate of from about 20% to 35% for conversion of a naphtha feed to aromatics at 50 hours time on stream for a 0.75 hydrogen/hexane stream at a temperature of about 515° C., ambient pressure, and a liquid hourly space velocity of about 8.6 per hour.

Aspect 26. The catalyst of any of aspects 1A-21, wherein the catalyst provides an isomerization of at least 10% at 50 hours time on stream for a 0.75 hydrogen/hexane stream at a temperature of about 515° C., ambient pressure, and a liquid hourly space velocity of about 8.6 h⁻¹.

Aspect 27. The catalyst of any of aspects 1A-21, wherein the catalyst provides an isomerization of from about 10 to about 25 for a 0.75 hydrogen/hexane stream at a temperature of about 515° C., ambient pressure, and a liquid hourly space velocity of about 8.6 h⁻¹.

Aspect 28. The catalyst of any of aspects 1A-21, where at least a portion of the alkanes have fewer than six linear atoms.

Aspect 29A. A method of aromatization of hydrocarbons, the method comprising contacting a feedstream with at least one catalyst, wherein the feedstream comprises an alkane, wherein the catalyst comprises an aluminum-silicon-germanium zeolite at which platinum has been deposited, wherein the aluminum-silicon-germanium zeolite has undergone a first ion exchange and contact with a second ion; and recovering an aromatic product.

Aspect 29B. A method of aromatization of hydrocarbons, the method consisting of contacting a feedstream with at least one catalyst, wherein the feedstream comprises an alkane, wherein the catalyst comprises an aluminum-silicon-germanium zeolite at which platinum has been deposited, wherein the aluminum-silicon-germanium zeolite has undergone a first ion exchange and contact with a second ion; and recovering an aromatic product.

Aspect 29C. A method of aromatization of hydrocarbons, the method consisting essentially of contacting a feedstream with at least one catalyst, wherein the feedstream comprises an alkane, wherein the catalyst comprises an aluminum-silicon-germanium zeolite at which platinum has been deposited, wherein the aluminum-silicon-germanium zeolite has undergone a first ion exchange and contact with a second ion; and recovering an aromatic product.

Aspect 30A. A method of making a catalyst, the method comprising: contacting an aluminum-silicon-germanium zeolite with a first ion to provide a first ion-exchanged zeolite, wherein the first ion comprises an alkali metal or an ammonium ion; contacting the first ion-exchanged zeolite with a second ion to provide a second ion-exchanged zeolite; and contacting the second ion-exchanged zeolite with at least a noble metal ion.

Aspect 30B. A method of making a catalyst, the method consisting essentially of: contacting an aluminum-silicon-germanium zeolite with a first ion to provide a first ion-exchanged zeolite, wherein the first ion comprises an alkali metal or an ammonium ion; contacting the first ion-exchanged zeolite with a second ion to provide a second ion-exchanged zeolite; and contacting the second ion-exchanged zeolite with at least a noble metal ion.

Aspect 30C. A method of making a catalyst, the method consisting of: contacting an aluminum-silicon-germanium zeolite with a first ion to provide a first ion-exchanged zeolite, wherein the first ion comprises an alkali metal or an ammonium ion; contacting the first ion-exchanged zeolite with a second ion to provide a second ion-exchanged zeolite; and contacting the second ion-exchanged zeolite with at least a noble metal ion.

Aspect 31. The method of aspect 30, wherein the second ion comprises an alkali metal ion.

Aspect 32. The method of aspect 30, wherein the first ion comprises cesium ion or ammonium ion.

Aspect 33. The method of aspect 30, wherein the second ion comprises sodium ions.

Aspect 34. The method of any of aspects 30-33, wherein the noble metal ion comprises platinum.

Aspect 35. The method of aspect 30, wherein the contacting the second ion-exchanged zeolite with at least a first metal ion comprises a process of ion-exchange, incipient wetness impregnation, or dry impregnation.

Aspect 36A. A catalyst consisting of: A zeolite comprising a framework and further comprising silicon, aluminum, and germanium and having a noble metal deposited at the zeolite, wherein the zeolite has undergone at least a first ion exchange and a second ion contact, wherein the catalyst is suitable for facilitating aromatization of alkanes to aromatics, and wherein the alkanes feed have five to twelve carbons and wherein at least a portion of the alkanes include isomers.

Aspect 36B. A catalyst consisting essentially of: A zeolite comprising a framework and further comprising silicon, aluminum, and germanium and having a noble metal deposited at the zeolite, wherein the zeolite has undergone at least a first ion exchange and a second ion contact, wherein the catalyst is suitable for facilitating aromatization of alkanes to aromatics, and wherein the alkanes feed have five to twelve carbons and wherein at least a portion of the alkanes include isomers.

Aspect 36C. A catalyst consisting of: A zeolite comprising a framework and further comprising silicon, aluminum, and germanium and having a noble metal deposited at the zeolite, wherein the zeolite has undergone at least a first ion exchange and a second ion contact, wherein the catalyst is suitable for facilitating aromatization of alkanes to aromatics, and wherein the alkanes feed have five to twelve carbons and wherein at least a portion of the alkanes include isomers.

Aspect 37. The catalyst of aspects 36A-C, where at least a portion of the alkanes have fewer than six linear carbon atoms.

Aspect 38A. A method of aromatization and isomerization of hydrocarbons, the method comprising contacting a feedstream with at least one catalyst, wherein the feedstream comprises an alkane, wherein the catalyst comprises an aluminum-silicon-germanium zeolite at which platinum has been deposited, the aluminum-silicon-germanium zeolite has undergone a first ion exchange and a second ion contact; and recovering an aromatic product.

Aspect 38B. A method of aromatization and isomerization of hydrocarbons, the method comprising consisting essentially of a feedstream with at least one catalyst, wherein the feedstream comprises an alkane, wherein the catalyst comprises an aluminum-silicon-germanium zeolite at which platinum has been deposited, the aluminum-silicon-germanium zeolite has undergone a first ion exchange and a second ion contact; and recovering an aromatic product.

Aspect 38C. A method of aromatization and isomerization of hydrocarbons, the method consisting of contacting a feedstream with at least one catalyst, wherein the feedstream comprises an alkane, wherein the catalyst comprises an aluminum-silicon-germanium zeolite at which platinum has been deposited, the aluminum-silicon-germanium zeolite has undergone a first ion exchange and a second ion contact; and recovering an aromatic product.

Aspect 39A. A method of aromatization of hydrocarbons, the method comprising contacting a feedstream with at least one catalyst, wherein the feedstream comprises an alkane, wherein the alkane comprises fewer than six linear carbon atoms, and wherein the catalyst comprises an aluminum-silicon-germanium zeolite at which platinum has been deposited, the aluminum-silicon-germanium zeolite has undergone a first ion exchange and a second ion contact; and recovering an aromatic product and wherein the catalyst facilitates aromatization and isomerization of the alkane comprising fewer than six linear carbon atoms.

Aspect 39B. A method of aromatization of hydrocarbons, the method consisting essentially of contacting a feedstream with at least one catalyst, wherein the feedstream comprises an alkane, wherein the alkane comprises fewer than six linear carbon atoms, and wherein the catalyst comprises an aluminum-silicon-germanium zeolite at which platinum has been deposited, the aluminum-silicon-germanium zeolite has undergone a first ion exchange and a second ion contact; and recovering an aromatic product and wherein the catalyst facilitates aromatization and isomerization of the alkane comprising fewer than six linear carbon atoms.

Aspect 39C. A method of aromatization of hydrocarbons, the method consisting of contacting a feedstream with at least one catalyst, wherein the feedstream comprises an alkane, wherein the alkane comprises fewer than six linear carbon atoms, and wherein the catalyst comprises an aluminum-silicon-germanium zeolite at which platinum has been deposited, the aluminum-silicon-germanium zeolite has undergone a first ion exchange and a second ion contact; and recovering an aromatic product and wherein the catalyst facilitates aromatization and isomerization of the alkane comprising fewer than six linear carbon atoms.

Aspect 40A. A catalyst comprising: A zeolite comprising a framework, the framework comprising silicon and aluminum, and a noble metal, wherein (a) the zeolite has undergone at least a first exchange with a first cation, wherein the first cation comprises a Group I or II cation or an ammonium ion and (b) the zeolite thereafter is contacted with a Group I or II cation, and wherein the step of contacting comprises an exchange, incipient wetness, or dry impregnation.

Aspect 40B. A catalyst consisting essentially of: A zeolite comprising a framework, the framework comprising silicon and aluminum, and a noble metal, wherein (a) the zeolite has undergone at least a first exchange with a first cation, wherein the first cation comprises a Group I or II cation or an ammonium ion and (b) the zeolite thereafter is contacted with a Group I or II cation, and wherein the step of contacting comprises an exchange, incipient wetness, or dry impregnation.

Aspect 40C. A catalyst consisting of: A zeolite comprising a framework, the framework comprising silicon and aluminum, and a noble metal, wherein (a) the zeolite has undergone at least a first exchange with a first cation, wherein the first cation comprises a Group I or II cation or an ammonium ion and (b) the zeolite thereafter is contacted with a Group I or II cation, and wherein the step of contacting comprises an exchange, incipient wetness, or dry impregnation.

Aspect 41. The catalyst of aspects 40A-40C, wherein the catalyst is suitable for aromatization and/or isomerization of alkanes and wherein at least a portion of the alkanes have fewer than six linear carbon atoms.

The disclosure having been generally described, the following examples are given as particular embodiments of the disclosure and to demonstrate the practice and advantages thereof. It is understood that the examples are given by way of illustration and are not intended to limit the specification or the aspects to follow in any manner.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. Unless indicated otherwise, percentages referring to composition are in terms of wt. %.

Only reasonable and routine experimentation will be required to optimize such process conditions. Though the following is illustrated using example values, processes, and materials, it is contemplated that other materials, processes, and values may be used according to design specifications.

Catalysts A, B, C, D, and E were prepared by modifying a GeZSM-5 zeolite with alkali metals.

Synthesis of Ge-ZSM-5 Zeolite. Solution #1 was made by diluting 15.84 grams (g) of 50 wt. % sodium hydroxide NaOH solution with 131.25 g of deionized (DI) water and subsequently dissolving 7.11 g of germanium dioxide. Solution #2 is made by diluting 3.84 g of sodium aluminate solution (23.6 wt. % alumina and 19.4 wt. % sodium oxide) with 153.9 g DI water. Solution #1 was added to 150 g of Ludox™ AS-40 (40 wt. % silica in a colloidal state) and vigorously stirred for 10 minutes to obtain a homogeneous mixture. Solution #2 was stirred into this mixture. After 15 minutes of vigorous agitation, 105.42 g of tetra-n-propyl ammonium hydroxide (TPAOH) was added and the mixture was stirred for 60 minutes. Finally, 23.32 g of glacial acetic acid was added to the gel to adjust the pH of the mixture to about 9. This mixture was loaded into a 1 L stainless steel autoclave and heated at 160° C. for 36 hours with stirring. Subsequently, the solids obtained were filtered from the mother liquor and washed with DI water. The solid was calcined at 550° C. for 10 hours in an oven with air flow. The MFI structure of the solid was confirmed by measuring the powder X-ray diffraction pattern.

Catalyst A. An amount of 8 grams (g) of GeZSM-5 was washed with 400 milliliters ml of aqueous cesium nitrate CsNO₃ (0.5 molar, M) then filtered. The filtrate was then rewashed three more times with 0.5 M CsNO₃ then washed in 200 ml of distilled water and filtered, totally four times. The zeolite powder was then calcined for three hours at 280° C. in air. Incipient wetness impregnation was carried out by adding, dropwise, a solution of 0.106 g of tetraamineplatinum (II) nitrate Pt(NH₂)₄(NO₃)₂ dissolved in 2.90 g of deionized water to 5.053 g of Cs-exchanged Ge ZSM-5. The material was dried for one hour at 120° C. in a drying oven then calcined at 280° C. for 3 hours. Elemental analysis gave 39.9 wt. % Si, 0.7 wt. % Al, 4.17 wt. % Ge, 5.0 wt. % cesium Cs, and 0.9 wt. % Pt.

Catalyst B. An amount of 0.035 g of sodium nitrate NaNO₃ was dissolved in 3.0 ml of deionized water. This solution was then added dropwise on the powder of NaCsGeZSM-5. The material was dried at 110° C. for one hour in air in a drying oven and then calcined at 520° C. for eight hours in air. Incipient wetness impregnation was carried out by adding drop wise a solution of 0.087 g Pt(NH₂)₄(NO₃)₂ dissolved in 4.0 g of deionized water to 3.904 g of the NaCs-exchanged ZSM-5. The material was dried for 1 hour in a 110° C. drying oven and then calcined at 280° C. for three hours. Elemental analysis gave 40.4 wt. % Si, 0.6 wt. %Al, 3.9 wt. % Ge, 0.6 wt % Na and 1.1 wt. % Pt.

Catalyst C. A 30.036 g amount of GeZSM-5 was stirred with 1500 ml of aqueous CsNO₃ (0.5M) at room temperature for two hours and then filtered. This step was repeated three more times. The material was then washed with 1500 ml of deionized water four times and dried at room temperature overnight. A 9.00 g amount of CsGeZSM-5 was stirred in 450 ml of aqueous NaNO₃ (0.5M) then filtered. This step was repeated four more times and the material was washed four times with 400 ml deionized water. The material was dried for 1 hour in a 110° C. drying oven then calcined at 280° C. for 3 hours. Incipient wetness impregnation was carried out by adding drop wise a solution of 0.083 g Pt(NH₂)₄(NO₃)₂ dissolved in 2.24 g of deionized water to 4.010 g of the NaCs-exchanged Ge-ZSM-5. The material was dried for 1 hour in a 110° C. drying oven then calcined at 280° C. for 3 hours. Elemental analysis gave 40.8 wt. % Si, 0.7 wt. % Al, 3.7 wt. % Ge, 0.5 wt % Na, 0.02 wt. % Cs, and 1.0 wt. % Pt.

Catalyst D. A 30.021 g amount of GeZSM-5 was stirred with 1500 ml of aqueous CsNO₃ (0.5M) at room temperature for two hours and then filtered. This step was repeated three more times. The material was then washed with 1500 ml of deionized water four times and dried for 1 hour in a 110° C. drying oven then calcined at 280° C. for 3 hours. 17.014 g of NaNO₃ was dissolved in 400 ml DI water and stirred well at room temperature. 8.068 g of CsGeZSM-5 was added to this solution and stirred for two hrs at room temperature. The slurry was filtered. The addition of NaNO₃ step was repeated three more times. The material was then washed with 1500 ml of DI water four times and dried for 1 hour in a 110° C. drying oven then calcined at 280° C. for three hrs. Incipient wetness impregnation was carried out by adding drop wise a solution of 0.144 g Pt(NH₂)₄(NO₃)₂ dissolved in 3.90 g of deionized water to 7.027 g of the NaCs-exchanged ZSM-5. The material was dried for 1 hour in a 110° C. drying oven then calcined at 280° C. for 3 hours. Elemental analysis gave 40.3 wt. % Si, 0.7 wt. % Al, 4.0 wt % Ge, 0.6 wt % Na 0.02 wt. % Cs, and 1 wt. % Pt.

Catalyst E. Ge-ZSM-5 (SiO₂/Al₂O₃=113 mol.; Si/Ge=21.6 mol.) was ion exchanged with 0.5 M ammonium chloride NH₄Cl solution at weight ratio solution/sample=20 at RT with stirring in four steps. Material was washed with D.I. water, dried overnight and calcined in air at 500° C. for 5 hours at heating rate 2° C./min. A 2.5 gram amount of ion-exchanged calcined Ge-ZSM-5 sample was impregnated under vacuum with 0.0572 g of NaNO₃ in 10 g of deionized water inside of 100 ml round bottom flask attached to a rotary evaporator and immersed into boiling water bath. The sample was dried at 90° C. overnight and calcined in air at 500° C. for 3 hour at heating rate 5° C./min. A 2.46 g amount of Na—Ge-ZSM-5 sample was impregnated with 0.0529 g of (NH₃)₄Pt(NO₃)₂ in 4 g of deionized water inside of 100 ml round bottom flask attached to rotary evaporator and immersed into preheated water bath (65-70° C.). Sample was stirred for about 5 minutes, then vacuum was applied and water was evaporated in about 35 minutes. The sample was dried at 90° C. overnight and calcined in air at 280° C. for three hours at heating rate 3° C./min. Elemental analysis gave 40.6 wt. % Si, 0.8 wt. % Al, 4.7 wt % Ge, 0.5 wt % Na and 1.1 wt. % Pt.

Catalyst Testing. Catalysts prepared by the procedures above were tested as follows: 20-40 mesh (420 μm-840 μm) catalyst particles, mixed with 20-40 mesh inert silica carbide (SiC) chips, were loaded into a 0.25 inch outer diameter (OD) plug flow reactor. N-hexane was vaporized into a stream of flowing hydrogen at a temperature of approximately 150° C. The liquid hourly space velocity (LHSV) was maintained at about 8.6 h⁻¹, with hydrogen to n-hexane molar ratio at 0.75. This gas mixture passed through the reactor, which was maintained at the specified reaction temperature, that is, around 515° C., by an external heating jacket.

The reaction products were analyzed by a gas chromatography. Products ranging in size from methane to dimethylnaphthalene were observed. The conversion (X) is defined as the molar fraction of the sum of benzene, toluene, xylenes, and ethyl benzene, all C₁-C₅ and C₇₊ materials produced. This conversion is presented on a molar C₆ basis, and being converted with their volume change factors as to the feed composition. The selectivities (S) reported are calculated as the sum of benzene, toluene, xylenes, and ethyl benzene produced divided by the sum of benzene, toluene, xylenes, and ethyl benzene and all C₁-C₅ and C₇₊ materials recovered. A variety of C₆ isomerization products were observed, including isohexanes (for example, 2-methylpentane) and their olefins (for example, 2-methylpentene). The isomerization (I) is the molar fraction of the sum of 2-methylpentane, 3-methylpentane, 2,2-dimethylbutane, 2,3-dimethylbutane, 2-methylpentene, 3-methylpentene, 2,2-dimethylbutene, 2,3-dimethylbutene, and iso-C₆ dienes. Values for conversion (X₅₀), selectivity (S₅₀), and isomerization (I₅₀) at 50 hours time on stream are presented in Table 1.

TABLE 1
Catalyst, formation conditions, and performance

| Catalyst | Catalyst description | X₅₀ | S₅₀ | I₅₀ |
|---|---|---|---|---|
| A | Pt/Cs—Ge-ZSM-5 | 22 | 94 | 2.6 |
| (Comparative) | | | | |
| B | Pt/Na/CsGe-ZSM-5 | 32 | 95 | 6 |
| | (Na impregnation on | | | |
| | Na back ion exchanged) | | | |
| C | Pt/Na—Cs—Ge-ZSM-5 | 22 | 94 | 21 |
| | (Na back ion-exchange 1) | | | |
| D | Pt/Na—Cs—Ge-ZSM-5 | 29 | 94 | 14 |
| | (Na back ion-exchange 2) | | | |
| E | Pt/Na/Ge-ZSM-5 (Na- | 28 | 94 | 19 |
| | only via impregnation) | | | |

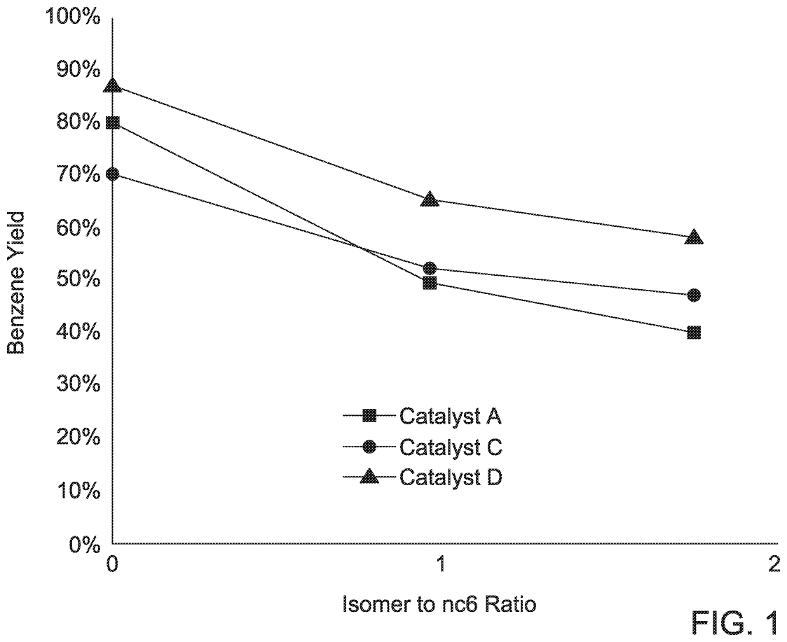

Catalysts A, C, and D were also evaluated under varying iso-hexane and n-hexanes amounts ranging from 100% n-hexane to 36% n-hexane and 64% iso-hexanes, at an liquid hourly space velocity LHSV of 2 h⁻¹; temperature at 535° C. The results of these experiments are shown in FIG. 1. FIG. 1 presents the benzene yield at different the isomer to n-hexane (C₆) ratio. The importance of the relative rates of isomerization and aromatization are clear based on the figure. Catalyst A shows the lowest benzene yield at higher iso-hexanes concentrations, while the catalyst with improved isomerization activity results in higher yields. Finally, Catalyst D that which had higher isomerization and aromatization activities showed the highest overall yields across all compositions tested.

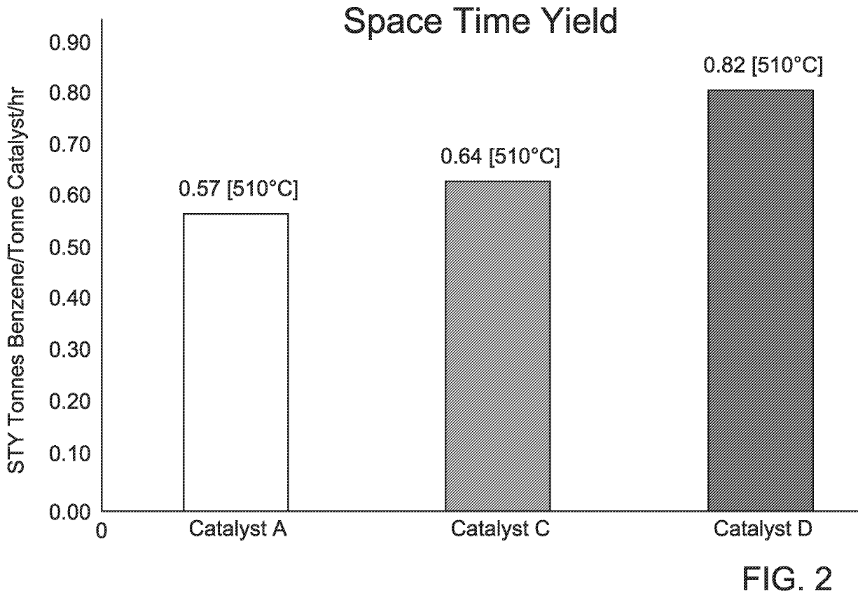

The overall space time yield (ton of benzene per ton of catalyst per hour) for Catalysts A, C, and D is shown in FIG. 2 at an LHSV of 2 h⁻¹; temperature, 510° C.; and an iso-hexane to normal-hexane ratio, 1.75. As the isomerization and aromatization activities are each subsequently increased, the overall space time yield of benzene increases accordingly.

As the isomerization and aromatization activities are each subsequently increased, the overall space time yield of benzene increases accordingly.

### Definitions

It is also to be understood that the terminology used herein is for the purpose of describing particular aspects only and is not intended to be limiting. As used in the specification and in the claims, the term “comprising” can include the embodiments “consisting of” and “consisting essentially of.” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used herein, the term “combination” is inclusive of blends, mixtures, alloys, reaction products, and the like.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

As used herein, the terms “optional” or “optionally” means that the subsequently described event or circumstance can or cannot occur, and that the description includes instances where said event or circumstance occurs and instances where it does not. For example, the phrase “optional additional additives” means that the additional additives can or cannot be included and that the description includes compositions that both include and do not include additional additives.

References in the specification and concluding claims to parts by weight of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent of a component, unless specifically stated to the contrary, is based on the total weight of the formulation or composition in which the component is included. As used herein the terms “weight percent,” “wt %,” and “wt. %,” which can be used interchangeably, indicate the percent by weight of a given component based on the total weight of the composition, unless otherwise specified. That is, unless otherwise specified, all wt % values are based on the total weight of the composition. It should be understood that the sum of wt % values for all components in a disclosed composition or formulation are equal to 100.

Unless otherwise stated to the contrary herein, all test standards are the most recent standard in effect at the time of filing this application. Each of the materials disclosed herein are either commercially available and/or the methods for the production thereof are known to those of skill in the art. It is understood that the compositions disclosed herein have certain functions. Disclosed herein are certain structural requirements for performing the disclosed functions and it is understood that there are a variety of structures that can perform the same function that are related to the disclosed structures, and that these structures will typically achieve the same result.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise. The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. While “about” permits some tolerance, a person of ordinary skill in the art would read the specification in light of his knowledge and skill for guidance on the level of that tolerance, and be reasonably apprised to a reasonable degree the metes and bounds of the claims.

In this document, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, the term aromatization refers to a catalytic process of converting alkanes, particularly light alkanes, into aromatics. Light alkane aromatization may provide higher value aromatic hydrocarbons such as benzene, toluene, and xylene.

The term “catalyst” means a substance that alters the rate of a chemical reaction. “Catalytic” means having the properties of a catalyst.

The term “conversion” means the mole fraction (i.e., percent) of a reactant converted to a product or products. As used herein, the term “selectivity” refers to the percent of converted reactant that went to a specified product, for example, 1-butene selectivity is the % of butane that converted to 1-butene.

As used herein, the term “about” or “approximately” are defined as being close to as understood by one of ordinary skill in the art. In one non-limiting aspect, the terms are defined to be within 10%, preferably within 5%, more preferably within 1%, and most preferably within 0.5%.

The term “substantially” and its variations are defined to include ranges within 10%, within 5%, within 1%, or within 0.5%.

All publications mentioned herein are incorporated herein by reference to disclose and describe the methods and/or materials in connection with which the publications are cited. The above description is intended to be illustrative, and not restrictive. For example, the above-described examples (or one or more aspects thereof) may be used in combination with each other. Other embodiments can be used, such as by one of ordinary skill in the art upon reviewing the above description. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the disclosure should be determined with reference to the appended claims, along with the full scope of equivalents to which such claims are entitled.

While typical aspects have been set forth for the purpose of illustration, the foregoing descriptions should not be deemed to be a limitation on the scope herein. Accordingly, various modifications, adaptations, and alternatives can occur to one skilled in the art without departing from the spirit and scope herein.

It will be apparent to those skilled in the art that various modifications and variations can be made in the present disclosure without departing from the scope or spirit of the disclosure. Other embodiments of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A catalyst comprising a zeolite comprising a framework, the framework comprising silicon, aluminum and a noble metal,
wherein (a) the zeolite has undergone at least a first exchange with a first Group I or II cation or a first ammonium cation and (b) the exchanged zeolite is contacted with a second Group I or II cation, wherein the first Group I or II cation is different from the second Group I or II cation,
wherein the step of contacting comprises an exchange, incipient wetness, or dry impregnation,
and wherein the catalyst provides an isomerization of at least 10% at 50 hours time on stream for a 0.75 hydrogen/hexane stream at a temperature of about 515° C., ambient pressure, and a liquid hourly space velocity of about 8.6 h⁻¹, and such that the isomerization is greater than that of a comparative catalyst having a same framework that has not undergone at least a first exchange and contacting.

2. The catalyst of claim 1 wherein the framework further comprises a metalloid selected from Group IV, Group V, Group XIII, Group XIV, Group V, or a combination thereof.

3. The catalyst of claim 1, wherein the framework further comprises germanium.

4. The catalyst of claim 3, wherein the zeolite comprises from about 0.2 wt. % to about 10 wt. % germanium.

5. The catalyst of claim 1, wherein the zeolite is MFI, BEA, MOR, or LTL.

6. The catalyst of claim 1, wherein the zeolite is ZSM-5.

7. The catalyst of claim 1, wherein the Group I or II cation is cesium.

8. The catalyst of claim 1, wherein the Group I or II cation is sodium.

9. The catalyst of claim 1, wherein the zeolite exhibits a molar ratio of alkali metal to aluminum in the framework of from 0.7 to 1.5 as a result of contacting the zeolite with a Group I or II cation in step (b).

10. The catalyst of claim 1, wherein more than 50% of the Group I or II cation is replaced by the second Group I or II cation when the zeolite thereafter is contacted with the second Group I or II cation in step (b).

11. The catalyst of claim 1, wherein the noble metal comprises platinum.

12. The catalyst of claim 1, wherein the noble metal is present in an amount of from 0.02 wt. % to 2 wt. % based on the total weight of the catalyst.

13. The catalyst of claim 1, wherein the catalyst catalyzes aromatization and/or isomerization of alkanes and wherein at least a portion of the alkanes have fewer than six linear carbon atoms.

14. A method of making a catalyst, the method comprising:
(a) contacting an aluminum-silicon-germanium zeolite with a first ion to provide a first ion-exchanged zeolite, wherein the first ion comprises a Group I or Group II cation or an ammonium ion;
(b) contacting the first ion-exchanged zeolite with a second ion to provide a second ion-containing zeolite; and
(c) contacting the second ion-containing zeolite with at least a noble metal ion.

15. The method of claim 14, wherein the zeolite exhibits a molar ratio of Group I or Group II cation to aluminum in the framework of from 0.7 to 1.5 as a result of contacting the zeolite with a Group I or II cation.

16. The method of claim 14, wherein more than 50% of the Group I or II cation is replaced by the second ion when the zeolite thereafter is contacted with the second ion.

17. The method of claim 14, wherein the contacting the first ion-exchanged zeolite with a second metal ion comprises a process of ion-exchange, incipient wetness impregnation, or dry impregnation.
